(19) United States
(12) Patent Application Publication
(10) Pub. No.: US 2014/0107429 A1
(43) Pub. Date: Apr. 17, 2014

(54) COMPUTER-EXECUTED METHOD, SYSTEM, AND COMPUTER READABLE MEDIUM FOR TESTING NEUROMECHANICAL FUNCTION

(71) Applicants: Charles A. Simkovich, Wexford, PA (US); Charles R. Myers, Wexford, PA (US)

(72) Inventors: Charles A. Simkovich, Wexford, PA (US); Charles R. Myers, Wexford, PA (US)

(21) Appl. No.: 14/008,963

(22) PCT Filed: Mar. 30, 2012
(86) PCT No.: PCT/US12/31515

§ 371 Date: Dec. 11, 2013

Related U.S. Application Data
(60) Provisional application No. 61/470,695, filed on Apr. 1, 2011.

(51) Int. Cl. A61B 5/16

(52) U.S. Cl. CPC A61B 5/16
USPC 600/300

(57) ABSTRACT

Described are methods, systems, and mediums for testing neuromechanical and neurocognitive function which reflect physical and mental compromise that may be associated with, for example, traumatic brain injury (TBI). In particular, a computer-executed method, system, and computer readable medium for testing neuromechanical and neurocognitive function of a subject are described which measure a subject's performance in a test comprising one or more test modules designed to challenge neuromechanical and neurocognitive function and compare the performance to one or more baselines to evaluate physical and mental compromise.

### FIELD OF THE INVENTION

The present invention generally relates to methods, systems, and mediums for testing neuromechanical function in addition to neurocognitive function which reflect physical and mental compromise that may be associated with, for example, traumatic brain injury (TBI) and/or other conditions. In particular, the present invention relates to a computer-executed method, system, and computer readable medium for testing neuromechanical and neurocognitive function of a subject which measure a subject's performance in a test comprising one or more test modules designed to challenge neuromechanical and neurocognitive function and compare the performance to one or more baselines to evaluate physical and mental compromise and/or improvement after any treatment.

### BACKGROUND OF THE INVENTION

In recent years, there has been increased interest in the development and use of computer-based tests specifically designed for sports-associated concussion management (Traumatic Brain Injury in Sports, an International Neuropsychological Perspective, 2004, Lovell et al., Eds., and Sports Medicine, 2005, Patel et al.). Examples of such tests include the Immediate Postconcussion Assessment and Cognitive Testing (IMPACT test), Axon Sport's Computerized Cognitive Assessment Tool (CCAT), and Headminder's Concussion Resolution Index (CRI). The advantages of computerized neurocognitive testing (CNT) include practical aspects, such as ease of administration, cost effectiveness, automated data collection, storage, and analysis, as well as psychometric aspects such as sensitivity to subtle cognitive deficits, availability of alternate forms of test batteries, and precise measurement of multiple domains of performance.

CNT has been used for athletes at the K-12, collegiate, and professional level. Typically, the athlete is assessed once while healthy in order to create a baseline neuropsychological profile. Following known trauma to the head, injured athletes are reassessed after physical and other reported symptoms have cleared to determine if cognition has returned to baseline levels. Physicians and sports medicine professionals utilize CNT results in addition to patient history and physical examination as the basis for return-to-play decisions.

Despite the remarkable potential offered by computerized neurocognitive testing and interest by clinicians in using it, several problems exist with current programs. The first of these is that the CNT data used as the basis for these medical judgments may be unreliable because both the baseline and post-injury neuropsychological assessments are performed by comparing the results of a single test when the results of both may be unrepresentative of the athlete's characteristic performance.

Unreliability of single-test results stems from the inherent variability of human performance. Any individual's performance on a CNT administered at a particular time can be influenced by many factors unrelated to injury, treatment, practice effects, or unreliability of the CNT itself. Such factors include, for example, fatigue, blood sugar levels, drug use, emotional stress, and motivation. These factors may or may not be discoverable, but even if discovered, their influence on results cannot be calculated or estimated with any degree of certainty. The results of a single CNT therefore may reflect performance levels that are potentially much higher or lower than the individual's normal performance capability. The only confident claim that can be made about the results of a single CNT is that they may be within that particular individual's normal performance range. Where the results fall within that range is unknown and cannot be determined from the results of a single CNT administration.

When evaluating an individual's return to baseline cognition levels, a typical approach is to calculate the difference between an individual's post-injury and baseline single test scores. But the difference between results at these two points in time will be influenced to some degree by the individual's natural performance variability. For example, if their baseline scores were taken when they were performing near the lower end of their “uninjured” range and their post-injury scores were near the upper end of their “injured” range, the difference between the two would be relatively small. If the circumstances were reversed (i.e., high baseline and low post-injury) the difference would be relatively large. In neither case would the results accurately reflect change due to recovery. In critical return-to-play decisions using data for which so much uncertainty exists is questionable, no matter what analysis is used.

Existing CNTs and their implementation approaches use various forms of population data (or sample data if population data are not available) as comparative standards due to the impossibility of calculating meaningful individual performance variation estimates using single data points. Normalizing an individual's CNT results based on their own performance variation is therefore not possible.

Using population or sample data as the basis for return-to-baseline evaluation is known to be problematic. For example, Iverson, Lovell, and Collins have stated, “The . . . reliable change estimate is optimized for the entire sample but is not as accurate for subsamples, such as the top 20%, middle 60%, and bottom 20% of scores.” See Iverson et. al., “Interpreting Change on ImPACT Following Sport Concussion” The Clinical Neuropsychologist, 2003, Vol. 17, No. 4, pp. 460-467. A single individual is the logical minimum subset of a population or sample, and the effects of the sub-optimization described by Iverson et. al. is particularly pronounced at that level because the variation of a population or sample can be roughly two to more than four times as great as that for an individual. Using a larger variation figure as the basis for return-to-baseline judgments results in lower normalized offsets from baseline values. Calculations made using larger variation values increase the possibility that baseline levels will be erroneously judged to have been restored, and consequently, that individuals who have not been restored to health will be returned to play prematurely.

A second potential problem with existing CNT instruments is that the test-retest reliability of several existing programs has been questioned. See Broglio et al., “Test-Retest Reliability of Computerized Concussion Assessment Programs,” J. Athletic Training, 2007, vol. 42, pgs. 509-514. Specifically, a study of healthy student volunteers who completed the ImPACT, Concussion Sentinel, and Concussion Resolution Index tests showed low to moderate test-retest reliability coefficients. A control test, the Memory and Concentration Test for Windows (MACT) was also administered to insure that the test results were not flawed due to sub-optimal effort. Since these three programs did not provide stable measures of cognitive functioning in healthy subjects, their utility in assessing post-concussive patients was questioned.

The United States military has attempted to use its CNT more extensively than commercial instruments that have targeted sports applications. The Automated Neuropsychological Assessment Metrics (ANAM) test is a computer-based tool developed by the U.S. Army to detect the speed and accuracy of attention, memory, and thinking ability. On May 28, 2008, a memorandum was issued by the Assistant Secretary of Defense making pre-deployment neurocognitive assessment mandatory for all service members. The baseline information collected in the assessment was then to be utilized in the event that a service member is injured in conflict, such as sustaining a concussion or traumatic brain injury in an explosion.

The ANAM test has been widely administered to military personnel prior to combat deployment and following traumatic brain injury in support of return-to-duty determinations, in similar fashion to application of commercial CNTs in sports. In addition, it has been administered to service members upon return from combat service in an attempt to detect cognitive indicators of possible unreported and/or undetected TBI.

The Department of Defense has become increasingly aware of the deficiencies of the ANAM test for these stated purposes. See Zwerdling et al., “Military's Brain-Testing Program a Debacle,” NPR Report, NPR.org, posted Nov. 28, 2011. A recent investigation has found that Pentagon's civilian leadership ignored years of warnings about the ANAM test. The military's highest ranking medical officials have said the test was flawed and no better than a “coin flip” (“Military Fails on Brain-Test Follow-ups,” Armytimes.com, posted Jun. 14, 2010). Recent research has also noted practice effects in five of six ANAM 4 subtests. Such practice effects must be taken into account or the results of subsequent assessments may be incorrectly attributed to patient improvement. (Eonta et al., “Automated Neuropsychological Assessment Metrics: Repeated Assessment with Two Military Samples,” Aviation, Space, and Environmental Medicine, 2011, vol. 82, pgs. 34-39).

CNT has been suggested for early detection of cognitive decline in the elderly. However, a systematic review of eleven test batteries appropriate to cognitive testing in the elderly showed great variability in manner of administration and wide variance in the level of rigor of validity testing (Wild et al., “The Status of Computerized Cognitive Testing in Aging: A Systematic Review,” Alzheimer's Dement., 2008, vol. 4, pgs. 428-437).

Accordingly, there exists a need for a computer-based testing method that accurately reflects physical and mental compromise and provides a prompt and sensitive indicator of existing or developing TBI, including concussions. Likewise, there exists a need for a computer-based method that provides an accurate indicator of physical and mental improvement in a subject after a TBI and any treatment thereof.

There also exists a need for a computer-based testing method that accurately reflects physical and mental compromise that may be associated with other impairments to brain function caused by, for example, drug use (prescription, non-prescription, or illicit), alcohol use/abuse, disease (dementia, Alzheimer's), depression, aging, fatigue, trauma suffered at birth, exposure to toxic chemicals, post-traumatic stress disorder and the like. Likewise, there exists a need for a computer-based method that provides an accurate indicator of physical and mental improvement or progression in a subject after treatment of an underlying condition.

A further need exists for a computer-based testing method that establishes statistically relevant subject baselines and population baselines to provide improved indicators of compromise and subsequent recovery.

### SUMMARY OF THE INVENTION

Briefly, the present invention is directed to computer-executed methods, systems, and computer readable mediums for testing neuromechanical function in addition to neurocognitive function, which reflect physical and mental compromise and/or improvement.

In accordance with various embodiments, the present invention comprises a computer-executed method for testing neuromechanical and neurocognitive function in a subject comprises: rendering a test comprising at least one test module for displaying the test to the subject; generating a request for subject input indicative of the subject's response to the displayed test; receiving the subject input indicative of the subject's response to the test; computing at least one subject score as a function of the received subject input; comparing the at least one subject score to at least one baseline comprising a subject baseline, wherein the subject baseline comprises a mean score and a standard deviation computed from a plurality of scores obtained from previously completed tests by the subject; and providing the at least one subject score and providing the score comparison for displaying to the subject.

In accordance with other embodiments, the present invention comprises a system for testing neuromechanical and neurocognitive function in a subject comprises: a memory storing instructions for a test comprising at least one test module, subject input, subject scores, and at least one subject baseline and a processor configured to execute computer executable instructions comprising: rendering a test comprising at least one test module for displaying a test to the subject; generating a request for subject input indicative of the subject's response to the displayed test; receiving the subject input indicative of the subject's response to the displayed test; computing at least one subject score as a function of the received subject input; comparing the at least one subject score to at least one subject baseline, wherein the at least one subject baseline comprises a mean score and a standard deviation computed from a plurality of scores obtained from previously completed tests by the subject; and providing the at least one subject score for displaying to the subject and providing the score comparison for displaying to the subject.

In accordance with further embodiments, the present invention comprises a computer-readable tangible non-transitory medium storing instructions for testing neuromechanical and neurocognitive function in a subject comprises computer executable instructions for: rendering a test comprising at least one test module for displaying the test to the subject; generating a request for subject input indicative of the subject's response to the displayed test; receiving the subject input indicative of the subject's response to the displayed test; computing at least one subject score as a function of the received subject input; comparing the at least one subject score to at least one subject baseline, wherein the at least one subject baseline comprises a mean score and a standard deviation computed from a plurality of scores obtained from previously completed tests by the subject; and providing the at least one subject score for displaying to the subject and providing the score comparison for displaying to the subject.

Other objects and features will be in part apparent and in part pointed out hereinafter.

### BRIEF DESCRIPTION OF THE DRAWINGS

FIG. 1 is a block diagram illustrating a system of one embodiment of the present invention.

FIG. 2 is a screen shot of a basketball game, which is an example of an interceptor test module.

FIG. 3 is a screen shot of a dodge ball game, which is an example of an avoidance test module.

FIG. 4 is a screen shot of a race track game, which is an example of a track test module.

FIG. 5A is a graph of a series of test scores illustrating a test score falling outside of three standard deviations of the baseline.

FIG. 5B is a graph of a series of test scores illustrating two test scores out of three falling outside of two standard deviations of the baseline.

FIG. 5C is a graph of a series of test scores illustrating four test scores out of five falling outside of one standard deviation of the baseline.

FIG. 5D is a graph of a series of test scores illustrating nine test scores on the same side of the baseline.

FIG. 5E is a graph of a series of test scores illustrating six test scores in a row in a decreasing trend.

FIG. 5F is a graph of a series of test scores illustrating six test scores in a row more than one standard deviation on either side of the baseline.

FIG. 5G is a graph of a series of test scores illustrating nine test scores in a row in an alternating trend.

FIG. 5H is a graph of a series of test scores illustrating 15 test scores in a row within one standard deviation of the baseline.

FIG. 6 is a block diagram of a series of test modules forming a test of one embodiment of the present invention as described in Example 1.

### DESCRIPTION OF THE PREFERRED EMBODIMENTS

In general, the present invention is directed to computer-executed methods, systems, and computer readable mediums for testing neuromechanical function in addition to neurocognitive function, which reflect physical and mental compromise and/or improvement.

In one aspect, the present invention is directed to a computer-executed method, system, and computer readable medium for testing neuromechanical and neurocognitive function which reflect physical and mental compromise associated with TBI and other closed cranial injuries. The present invention may be used effectively to screen for indicators of various levels of possible TBI (including mild TBI, or concussion), as well as other closed cranial injuries that do not qualify as concussions. Further, the present invention may be used effectively to track indicators of possible improvement of a subject after a TBI or other closed cranial injury and any subsequent treatment. Accordingly, the present invention provides an assessment tool for possible TBI and other possible closed cranial injury with improved sensitivity.

In another aspect, the present invention is directed to a computer-executed method, system, and computer readable medium for testing neuromechanical and neurocognitive function which reflect physical and mental compromise associated with possible impairment to brain function caused by, for example, drug use/abuse (prescription, non-prescription, or illicit), alcohol use/abuse, disease (e.g., dementia, Alzheimer's), depression, aging, fatigue, trauma, exposure to toxic chemicals, psychiatric disorders and the like. Further, the present invention may be used to effectively track possible improvement or recovery of a subject after any subsequent treatment of the underlying condition. Accordingly, the present invention provides an assessment tool for a wide-range of conditions that may affect brain function.

In still another aspect, the present invention is directed to a computer-executed method, system, and computer readable medium useful for testing neuromechanical and neurocognitive function of a subject prior to, during and/or subsequent to engaging in the subject's occupation. For example, in various embodiments, the present invention is useful for testing the neuromechanical and neurocognitive function of aircraft pilots, air traffic controllers, watercraft operators, truck drivers, train engineers, mass transit drivers, military personnel, law enforcement officers, security guards, and so on.

In yet another aspect, the present invention is directed to a computer-executed method, system, and computer readable medium for testing neuromechanical and neurocognitive function which provide for statistically meaningful indicators of performance. For example, establishing subject baselines based on the test scores obtained from a plurality of tests previously completed by the subject provides a more complete and representative description of a subject's characteristic performance. Further, test performance deviating from the subject baseline provides a more accurate indication of compromise and subsequent improvement after any treatment. Also, by establishing population baselines which are calculated based on the test scores of a host of subjects having similar attributes (e.g., age and/or gender), improved indicators of representative test performance and compromise are obtained.

In another aspect, the present invention provides computer-executed method, system, and computer readable medium for testing neuromechanical and neurocognitive function which includes a test or test modules that are customizable to the particular subject or group of subjects. Customization increases the relevancy of the test to a subject, thereby increasing the subject's interest in completing the test. Further, in various aspects, the present invention includes test modules that are games or game-like to enhance a subject's interest in completing the test.

### I. Traumatic Brain Injury (TBI) and Other Closed Cranial Injury

In various embodiments, the present invention provides for testing neuromechanical and neurocognitive function which reflects physical and mental compromise associated with TBI and other closed cranial injuries and/or recovery from these injuries. TBI and other closed cranial injuries may be suffered during the course of a variety of sports and activities such as football, hockey, boxing, rugby, lacrosse, baseball, gymnastics, cheerleading, martial arts, automobile or boat racing, and so on. TBI and other closed cranial injuries may also be suffered during a car or bike accident, a fall, a battery, trauma suffered at birth or any event during the course of life where an external force is applied to the cranium.

TBI and other closed cranial injuries may result in neurological, structural, muscular, and/or organic impairment. Neurological impairment includes impairment to the brain, nerves, and sensory organs. Cranial injury can impact any and all parts of the human nervous system, not just the brain. The sensory organs, particularly those residing in the head, may be affected, as well as the nerve pathways that link the sensory organs and the brain.

Structural impairment includes impairment to the cranium and meninges. When the head is struck by either an object or a concussive force, the cranial bones displace to absorb the shock and protect the brain. This displacement does not self-correct, and in their displaced condition the cranial bones no longer move as they should. Because of the very close tolerances within the skull, their displacement also negatively affects the meninges, adjacent brain tissue, general and localized blood flow to the brain, and cerebrospinal fluid (CSF) flow.

Muscular impairment includes impairment to the ocular and facial muscles. The ocular and facial muscles all attach to cranial bones. When intrinsic movement of these bones is compromised, the muscles attached to them are either stretched or compressed, and unable to function properly. The brain compensates for their compromised condition as best it can, but normal performance cannot be duplicated. In addition, the muscles tire quickly when they are compromised.

Organic impairment includes impairment due to lack of oxygenation, lack of nourishment, and impaired and/or reduced metabolic waste removal. Impaired cranial bone movement results in two conditions that affect the brain's ability to function properly. Ischemia (restricted blood supply) caused by impaired cranial bone movement reduces the oxygen and nutrient levels supplied to the brain. Reduced CSF flow limits the body's ability to remove metabolic wastes from around the brain cells. Over time, these two conditions progressively reduce the brain's ability to perform a wide range of functions.

As a result of these impairments, cognitive function (verbal and iconic recall) is often compromised.

Psychomotor function (eye tracking, hand-eye coordination) may be compromised as a result of these impairments. Extraocular muscle attachment to displaced cranial bones stretches some muscles and compresses others, resulting in predictable physiological compromise. Improper and ineffective eye tracking is indicative of these effects, with ancillary hand-eye coordination deficiencies.

Also, ocular function (fixation, smooth motion) may be compromised as a result of these impairments. The effect of cranial bone displacement on the extraocular muscles compromises the elevation, depression, adduction, abduction, intorsion, and extorsion functions of the eyes. Macro-level collateral compromise includes fixation on stationary objects and following moving objects.

Further, visual perception (tremor, drift, microsaccades) may be compromised as a result of these impairments. Micro-level collateral compromise includes the micro-level eye displacements that are essential for effective visual perception. Tremor, drift, and microsaccades prevent visual neural adaptation. When these movements are abnormal, the brain literally does not respond to stimuli transmitted from the retina via the optic nerve.

Left or right brain function may also be inhibited or over-stimulated from closed cranial injuries due to abnormal movement of the greater wings of the sphenoid bone. Decreased blood circulation to the frontal and mid brain caused by inferior movement of the frontal bone may also impair short-term memory.

Applicants have discovered that administration of a neuromechanical and neurocognitive test in accordance with the present invention which, among other things, challenges eye movement provides an immediate and sensitive indicator of TBI and other closed cranial injuries.

### II. Other Conditions

In various embodiments, the present invention provides for testing neuromechanical and neurocognitive function which reflects physical and mental compromise that may be associated with impairments to brain function caused by, for example, substance use or abuse, various diseases including nervous system diseases, disorders which alter brain activity, psychiatric disorders, conditions which impact brain function, or accidental or naturally-occurring events which impact brain function. Further, the present invention provides for effectively tracking improvement or recovery of a subject after any subsequent treatment of the underlying condition, symptoms, or cause.

Substance use/abuse includes various uses, over-uses and misuses (abuse) of prescription drugs, non-prescription drugs (over-the-counter drugs), illicit drugs (e.g. methamphetamines, cocaine, etc.), legalized intoxicants (e.g., alcohol) and/or unregulated substances. Substance use also includes chemotherapy for treating cancer. Substance use/abuse often impacts the normal function of the central nervous system. For example, alcohol intoxication impairs eye movement by slowing the function of the central nervous system and interfering with coordination of muscles throughout the body, including the ocular muscles. Drug use also impairs eye movement by altering the function of the central nervous system (inhibiting or over-stimulating depending on the type of drug) and interfering with cognition and muscle coordination throughout the body, including the ocular muscles.

Nervous system diseases include various diseases such as Parkinson's and Alzheimer's and other forms of dementia. These diseases typically result in the loss of brain and/or muscle function, which includes impairment to cognition, eye movement, and/or muscle coordination. Other diseases such as diabetes cause blurry vision, fatigue, and mental confusion, which interfere with cognition and ocular function.

Disorders which alter brain activity include, for example, convulsive disorders such as epilepsy. These disorders manifest symptoms including unpredictable seizures which interfere with coordination and/or control of muscles throughout the body, including the ocular muscles.

Psychiatric disorders include, for example, depression, post-traumatic stress disorder (PTSD), anxiety disorders (phobias), bipolar disorders, attention deficit disorder (ADHD), schizophrenia and the like. These disorders manifest symptoms including difficulty concentrating, fatigue, hyperactivity, memory loss, other cognitive impairment, and/or loss of motivation. These disorders or the symptoms of these disorders interfere with coordination and/or control of muscles throughout the body, including the ocular muscles.

Other conditions which impact brain function include aging, fatigue, insomnia, sleep deprivation, stress, and pain. For example, fatigue due to lack of sleep or exertion impairs eye movement by slowing the function of the central nervous system and interfering with cognition and coordination of muscles throughout the body, including the ocular muscles.

Accidental or naturally-occurring events which impact brain function include, for example, accidental or occupational exposure to toxic chemicals. These events can damage the central nervous system and interfere with cognition and coordination of muscles throughout the body, including the ocular muscles.

Applicants have found that administration of the neuromechanical and neurocognitive test in accordance with the present invention which, among other things, challenges eye movement provides an immediate and sensitive indicator of impairments to brain function caused by substance use/abuse (e.g., use and/or abuse of prescription, non-prescription, or illicit drugs and/or use/abuse of alcohol), nervous system diseases (e.g., Alzheimer's and Parkinson's) and other diseases, disorders which alter brain activity, psychiatric disorders, conditions which impact brain function, conditions which impact brain function (e.g., fatigue or aging), accidental or naturally-occurring events which impact brain function (e.g., exposure to toxic chemicals) and combinations thereof.

### III. Occupational/Clinical Uses

In various embodiments, the present invention provides for testing neuromechanical and optionally neurocognitive function which reflects the physical and mental readiness of a subject prior to, during, or subsequent to engaging in the subject's occupation. In some embodiments the present invention provides for testing at least neuromechanical function of a subject prior to, during, and/or subsequent to engaging in the subject's occupation.

In many occupations, unimpaired neuromechanical function is beneficial, if not entirely necessary, for effective job performance. For example, unimpaired neuromechanical function is essential for aircraft/spacecraft crew, air traffic controllers, watercraft operators, truck drivers, train engineers, mass transit drivers, military personnel, law enforcement officers, security guards, and so on in order to perform their jobs effectively and without harm to people and property. Thus, in various embodiments, the present invention may be used to evaluate the neuromechanical function of subjects prior to, during, or subsequent to the subjects engaging in their occupation. Such an assessment provides an employer or supervisor with valuable information to assist in determining whether employees are fit to perform their job tasks. Further, the assessment provides an employer or supervisor with an evaluation means that does not require determining the underlying cause of impairment.

In various embodiments, the present invention provides for testing neuromechanical and/or neurocognitive function of subjects participating in a clinical setting for the evaluation of a drug. The test can be used to track physical or mental compromise or any improvement thereof of the subjects during the trial.

### IV. Computer-Executed Method

Typically, the neuromechanical and neurocognitive test of the present invention is computer-based or web-based. Accordingly, in some embodiments, the present invention includes a computer-executed method. In various embodiments, the computer-executed method comprises: rendering a test comprising at least one test module for displaying the test to the subject; generating a request for subject input indicative of the subject's response to the displayed test; receiving the subject input indicative of the subject's response to the test; computing at least one subject score as a function of the received subject input; comparing the at least one subject score to at least one baseline comprising a subject baseline, wherein the subject baseline comprises a mean score and a standard deviation computed from a plurality of scores obtained from previously completed tests by the subject; and providing the at least one subject score and providing the score comparison for displaying to the subject.

In accordance with some embodiments of the present invention, the test or test modules may be customized based on subject-specific information received prior to rendering of the test. Therefore, in various embodiments, the computer-executed method comprises: generating a request for subject-specific information from the subject; receiving the subject-specific information; rendering a test comprising at least one test module based on the subject-specific information received for displaying to the subject; generating a request for subject input indicative of the subject's response to the test module; receiving subject input indicative of the subject's response to the test module; computing a subject score from as a function of the received subject input; comparing the subject score to one or more baselines; and providing the subject score and the score comparison for displaying to the subject.

The test may be displayed to a subject on a wide range of electronic display devices including, for example, a computer monitor, touch-screen, television, electronic tablet device, hand-held gaming device, and wireless phone. Further, subject input may be received by various input devices including, for example, a computer mouse, keyboard, touch-screen, touch-pad, joystick including an isometric joystick, remote control, a gaming controller, a motion sensing input device and combinations thereof.

### V. System

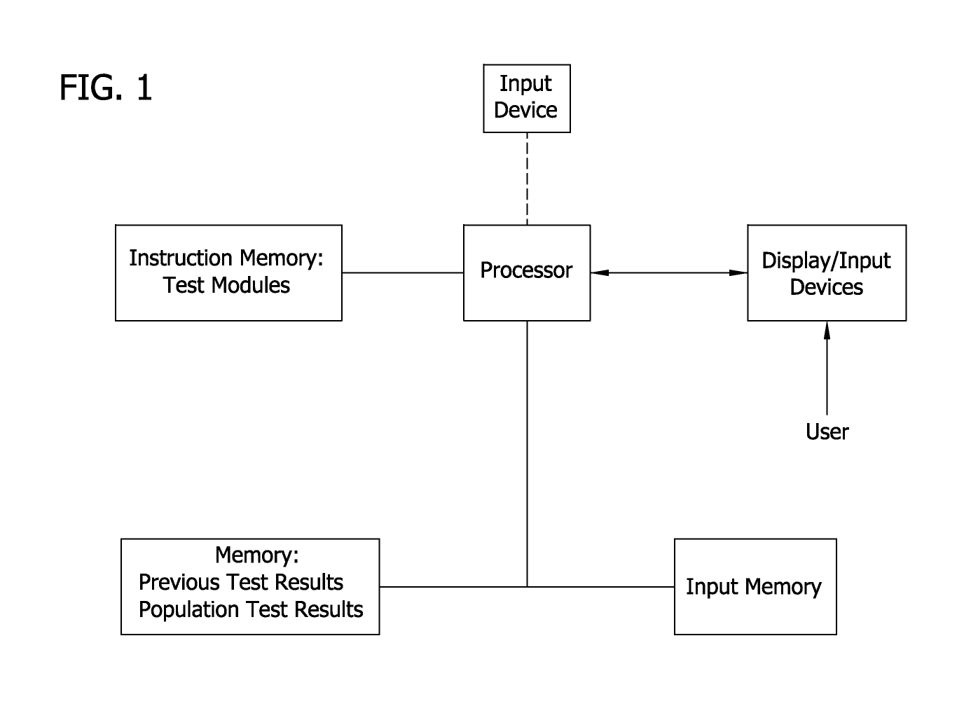

In accordance with various embodiments of the present invention, a system comprising a neuromechanical and neurocognitive test is provided. The present invention may be implemented on a variety of computing devices and systems, wherein these computing devices include the appropriate processing mechanisms and computer-readable media for storing and executing computer executable instructions, such as programming instructions, code, and the like. For example, as shown in FIG. 1, a system according to one embodiment of the present invention includes a processor that serves to execute computer executable instructions received in the appropriate data format. The system may include a variety of memory for storing instructions for the test and test modules, previous subject test results and population test results, input responses, and baselines.

In various embodiments, the present invention includes a system for testing neuromechanical function in a subject a memory storing instructions for a test comprising at least one test module, subject input, subject scores, and at least one subject baseline and a processor configured to execute computer executable instructions comprising: rendering a test comprising at least one test module for displaying the test to the subject; generating a request for subject input indicative of the subject's response to the displayed test; receiving the subject input indicative of the subject's response to the displayed test; computing at least one subject score as a function of the received subject input; comparing the at least one subject score to at least one subject baseline, wherein the at least one subject baseline comprises a mean score and a standard deviation computed from a plurality of scores obtained from previously completed tests by the subject; and providing the at least one subject score for displaying to the subject and providing the score comparison for displaying to the subject.

In some embodiments, the system for testing neuromechanical and neurocognitive function in a subject comprises a memory storing instructions for storing a test comprising at least one test module and a processor configured to execute computer executable instructions comprising: generating a request for subject-specific information from the subject; receiving the subject-specific information; rendering a test comprising at least one test module based on the subject-specific information received for displaying to the subject; generating a request for subject input indicative of the subject's response to the test module; receiving subject input indicative of the subject's response to the test module; computing a subject score as a function of the received subject input; comparing the subject score to one or more baselines; and providing the subject score and the score comparison for displaying to the subject.

The system may further comprise a variety of display devices. In some embodiments, the system may further comprise an electronic display device including, for example, a computer monitor, touch-screen, television, electronic tablet device, hand-held gaming device, or wireless phone.

A subject may enter inputs in response to one or more test modules using a variety of input devices. In various embodiments, the system may further comprise an input device including, for example, a computer mouse, keyboard, track-ball, touch-screen, touch-pad, joystick including an isometric joystick, remote control, a gaming controller, a motion sensing input device and combinations thereof.

### VI. Computer Readable Medium

In accordance with the present invention, a computer-readable tangible non-transitory medium storing instructions for testing neuromechanical and neurocognitive function in a subject is provided. In various embodiments, the computer-readable tangible non-transitory medium comprises instructions for: rendering a test comprising at least one test module for displaying the test to the subject; generating a request for subject input indicative of the subject's response to the displayed test; receiving the subject input indicative of the subject's response to the displayed test; computing at least one subject score as a function of the received subject input; comparing the at least one subject score to at least one subject baseline, wherein the at least one subject baseline comprises a mean score and a standard deviation computed from a plurality of scores obtained from previously completed tests by the subject; and providing the at least one subject score for displaying to the subject and providing the score comparison for displaying to the subject.

In some embodiments, the computer-readable tangible non-transitory medium comprises computer executable instructions for generating a request for subject-specific information from the subject; receiving the subject-specific information; rendering a test comprising at least one test module based on the subject-specific information received for displaying to the subject; generating a request for subject input indicative of the subject's response to the test module; receiving subject input indicative of the subject's response to the test module; computing a subject score as a function of the received subject input; comparing the subject score to one or more baselines; and providing the subject score and the score comparison for displaying to the subject.

### VII. Test and Test Modules

Generally, the computer-executed method, system, and computer readable medium of the present invention comprise a test (or memory storing computer executable instructions for a test or computer executable instructions for a test) comprising at least 2, at least 3, at least 4, at least 5, at least 6, at least 7, at least 8, at least 9, at least 10, at least 15, at least 20, or at least 25 test modules. In various embodiments, the test comprises from 2 to 50, from 5 to 40, from 10 to 30, or from 20 to 30 test modules.

As previously described, compromised/impaired neuromechanical function, in particular compromised/impaired eye movement, is an immediate and sensitive indicator of TBI and other closed cranial injuries, as well as other impairments to brain function substance use/abuse (e.g., use and/or abuse of prescription, non-prescription, or illicit drugs and/or use/abuse of alcohol), nervous system diseases (e.g., Alzheimer's and Parkinson's) and other diseases, disorders which alter brain activity, psychiatric disorders, conditions which impact brain function, conditions which impact brain function (e.g., fatigue or aging), accidental or naturally-occurring events which impact brain function (e.g., exposure to toxic chemicals) and combinations thereof. Accordingly, in various embodiments, the computer-executed method, system, and computer readable medium of the present invention comprise a test (or memory storing computer executable instructions for a test or computer executable instructions for a test) comprising at least 1, at least 2, at least 3, at least 4, at least 5, at least 6, at least 7, at least 8, at least 9, at least 10, at least 15, or at least 20 test modules that challenge eye movement. In some embodiments, the computer-executed method, system, and computer readable medium comprise a test comprising from about 2 to about 50, from about 5 to about 50, from about 10 to about 50, from about 20 to about 50, from about 10 to about 40, from about 20 to about 40, from about 30 to about 40, from about 15 to about 30, or from about 20 to about 30 (e.g., from about 20 to about 25) test modules that challenge eye movement.

The test modules that challenge eye movement may challenge saccadic eye movement (rapid parallel movement of the eyes between fixations), pursuit eye movement (slower parallel movement used—for example—in motion tracking), or both. Accordingly, in various embodiments, the test comprises at least 1, at least 2, at least 3, at least 4, at least 5, at least 6, at least 7, at least 8, at least 9, at least 10, or at least 15 test modules that challenge saccadic eye movement. In some embodiments, the test comprises at least 1, at least 2, at least 3, at least 4, at least 5, at least 6, at least 7, at least 8, at least 9, at least 10, or at least 15 test modules that challenge pursuit eye movement. In these and other embodiments, the test comprises at least 1, at least 2, at least 3, at least 4, at least 5, at least 6, at least 7, at least 8, at least 9, at least 10, or at least 15 test modules that challenge both saccadic eye movement and pursuit eye movement. Further, in various embodiments, the test modules challenge both eye movement and cognitive ability.

In accordance with various embodiments of the present invention, the test may comprise specific test modules that challenge eye movement. Accordingly, in various embodiments, the test comprises at least one test module for displaying to the subject selected from the group consisting of an interceptor test module, an avoidance test module, a track test module, and combinations thereof.

Interceptor Modules

Test modules that challenge eye movement, particularly saccadic and pursuit eye movements, include an interceptor test module. Accordingly, in various embodiments, the test comprises an interceptor test module. The interceptor test module comprises (1) a subject-controlled object having a display position that is movable in response to subject input and (2) a series of generated target objects which move about the visual field of a display starting from randomly selected positions, wherein the subject is requested to move the subject-controlled object to intercept (i.e., contact in a pre-defined area) as many target objects as possible in a pre-defined amount of time (e.g., about 45 or 60 seconds). The number of target objects intercepted by the subject-controlled object may be recorded for scoring purposes. In some embodiments, the number of target objects partially contacted by the subject-controlled object may also be recorded for scoring purposes.

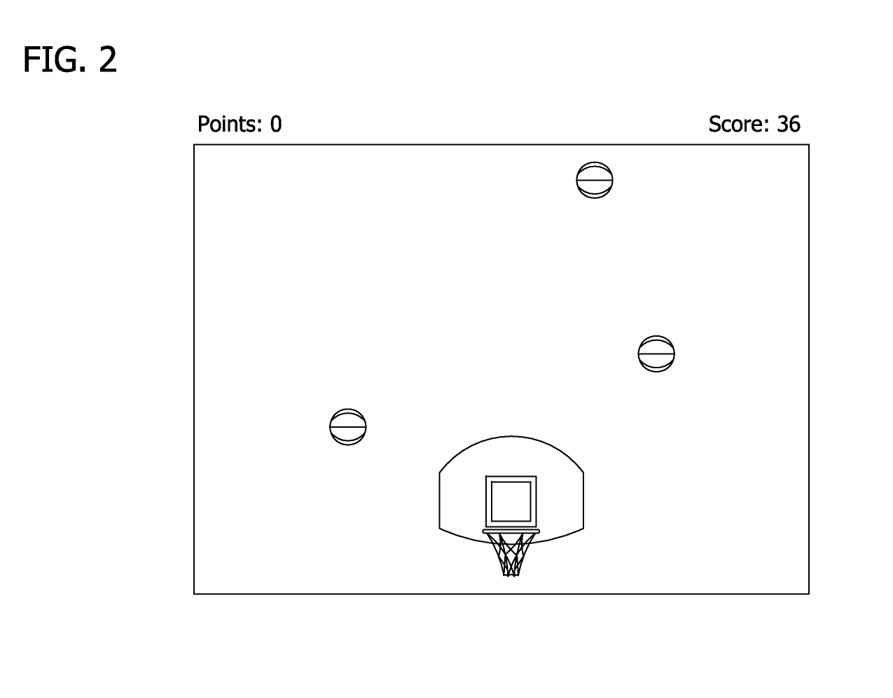

In various embodiments, a series of at least 50, 75, 100, 125, or 150 of generated target objects move across the visual field of the display. In various embodiments the generated target objects resemble a sports ball (e.g., basketball, football, baseball, volleyball, soccer ball, hockey puck, etc.) and the subject-controlled object resembles the corresponding sports goal, receptacle, or player (e.g., basketball goal, football receiver, baseball glove, volleyball player, soccer net, hockey goal, etc.). FIG. 2 shows a screen shot of a basketball game, which is an example of an interceptor test module. In a preferred embodiment, the generated targets (e.g., basketballs as shown in FIG. 2) move from the top of the visual field of the display to the bottom, and the subject-controlled object (e.g., a basketball goal as shown in FIG. 2) has a display position that is movable horizontally (side-to-side) across the visual field in response to subject input.

In these and other embodiments, the generated target objects and subject-controlled object are customized to a subject's sport or activity of interest.

Avoidance Modules

Other test modules that challenge eye movement, particularly saccadic and pursuit eye movements, include an avoidance test module. Accordingly, in various embodiments, the test comprises an avoidance test module. The avoidance test module comprises (1) a subject-controlled object having a display position that is movable in response to subject input and (2) a series of generated target objects which move across the visual field of a display starting from randomly selected positions, wherein the subject is requested to move the subject-controlled object to avoid contact with as many of the target objects as possible in a pre-defined amount of time (e.g., about 45 or 60 seconds). The number of times the subject-controlled object contacts a target object may be recorded for scoring purposes.

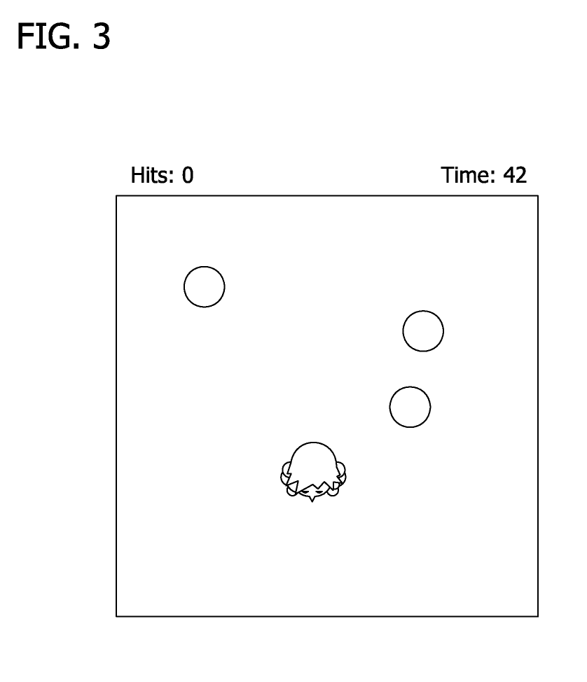

In various embodiments, a series of at least 50, 75, 100, 125, or 150 of generated target objects move across the visual field of the display. In various embodiments, at least a portion of the generated target objects start from each side (left, right, top, and bottom) of the visual field. In various embodiments, the generated target objects resemble balls (e.g., dodge balls) and the subject-controlled object resembles an animated person. FIG. 3 provides a screen shot of a dodge ball game, which is an example of an avoidance test module.

Track Modules

Further test modules that challenge eye movement, particularly pursuit and saccadic eye movements, include a track test module. Accordingly, in various embodiments, the test comprises a track test module. The track test module comprises (1) a pre-defined closed-loop course having inner and outer boundaries and comprising a plurality of linear and curved segments and (2) a subject-controlled object having a display position that is movable in response to subject input, wherein the subject is requested to move the subject-controlled object through the pre-defined closed-loop course as many times as possible in a pre-defined amount of time (e.g., about 45 or 60 seconds) while avoiding contact with the inner and outer boundaries of the course. If the subject-controlled object contacts the inner and outer boundaries of the course, then the object may be returned to the start of the course and/or incur a penalty. The number of segments of the course that are completed without contacting the inner and outer boundaries and number of times (laps) the course is completed may be recorded for scoring purposes. Also, the number of times the subject-controlled object contacts the inner and outer boundaries of the course may be recorded for scoring purposes.

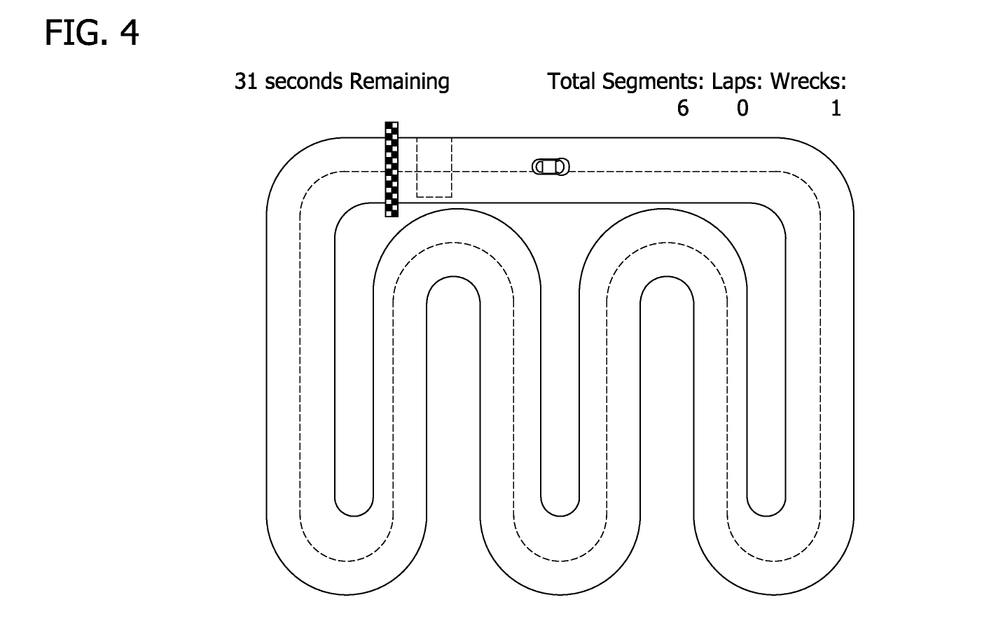

In various embodiments, the pre-defined closed-loop course resembles a race track and the subject-controlled object resembles a race car. FIG. 4 is a screen shot of a race track game, which is an example of a track test module.

In various embodiments, the pre-defined closed-loop course comprises at least 2, 3, 4, 5, 6, 7, 8, 9, or 10 curved segments (e.g., from about 5 to about 30, from about 10 to 25, or from about 10 to about 20 curved segments).

Object Recall Moving Display Test Modules

Other test modules that challenge eye movement, particularly pursuit and saccadic eye movements, include object recall moving display test modules. In these modules, eye movement and cognitive ability are challenged. Accordingly, in various embodiments, the test comprises one or more object recall moving display test modules. An object recall moving display test module comprises displaying a series of target objects to the subject, one object at a time, for a period of time (e.g., about 0.5, 1, 2, 3, 4, or 5 seconds). The target objects move across the visual field of the display in various pre-defined randomized patterns. For example, an object may move in a horizontal pattern (left to right or right to left), a vertical pattern (top to bottom or bottom to top), a diagonal pattern (in either direction from one corner of the visual field to another corner that is diagonal from the first corner), or a spiral pattern. Typically, no two objects move in the same pattern in a given test module. After the target objects have been initially displayed, the target objects along with one or more distracter (i.e., non-target) objects are displayed statically in a group wherein the subject is requested to select the target objects among the objects displayed. The number of target objects selected, total number of target objects selected, number of times target or distracter items are selected more than once, number of distracter objects selected, number of selections made on neither a target object nor a distracter object (i.e., stray selections), total number of selections made, total number of selections less than or exceeding the number required to select all the target objects once, and the total time to complete the recall portion of the module may be recorded for scoring purposes.

In various embodiments, the object recall moving display test module comprises at least 2, 3, 4, 5, 6, 7, 8, 9, 10, 11, 12, 13, 14, or 15 target objects. In some embodiments, the object recall moving display test module comprises 4, 5, 6, 7, 8, 9, or 10 target objects (e.g., 5 or 6 target objects). In various embodiments, the object recall moving display test module comprises at least 2, 3, 4, 5, 6, 7, 8, 9, 10, 11, 12, 13, 14, or 15 distracter objects. In some embodiments, the object recall moving display test module comprises 4, 5, 6, 7, 8, 9, or 10 distracter objects (e.g., 5 or 6 distracter objects). In some embodiments, the number of distracter objects matches the number of target objects.

In various embodiments, the target and distracter objects are selected from the group consisting of letters, numbers, words, figures, pictures, shapes, and combinations thereof. Accordingly, in various embodiments, the test comprises at least one word recall moving display test module, which comprises target words and distracter words. In other embodiments, the test comprises at least one figure recall moving display test module, which comprises target figures and distracter figures. In some embodiments, the test comprises at least one word recall moving display test module and at least one figure recall moving display test module.

Sequential Object Recall Moving Display Test Modules

Other test modules that challenge eye movement, particularly pursuit and saccadic eye movements, include sequential object recall moving display test modules. Similar to the object recall moving display test modules previously described, these modules challenge both eye movement and cognitive ability. Accordingly, in various embodiments, the test comprises one or more sequential object recall moving display test modules.

A sequential object recall moving display test module comprises displaying a series of target objects to the subject, one object at a time, for a period of time (e.g., about 0.5, 1, 2, 3, 4, or 5 seconds). The target objects move across the visual field of the display in various pre-defined randomized patterns. For example, an object may move in a horizontal pattern (left to right or right to left), a vertical pattern (top to bottom or bottom to top), a diagonal pattern (in either direction from one corner of the visual field to another corner that is diagonal from the first corner), or a spiral pattern. Typically, no two objects move in the same pattern in a given test module. After the target objects have been initially displayed, the target objects are displayed statically in a group wherein the subject is requested to select the target objects in the order they were presented. The order the objects are selected, the number of objects selected, total number of selections less than or exceeding the number required to select all the target objects once, number of selections not in the correct order, number of times target items are selected more than once, number of selections not made on an object (i.e., stray selections), total number of selections made, and the total time to complete the recall portion of the module may be recorded for scoring purposes.

In various embodiments, the sequential object recall moving display test module comprises at least 2, 3, 4, 5, 6, 7, 8, 9, 10, 11, 12, 13, 14, or 15 target objects. In some embodiments, the sequential object recall moving display test module comprises 4, 5, 6, 7, 8, 9, or 10 target objects (e.g., 5 or 6 target objects).

In various embodiments, the target objects are selected from the group consisting of letters, numbers, words, figures, pictures, shapes, and combinations thereof. Accordingly, in various embodiments, the test comprises at least one sequential word recall moving display test module, which comprises target words. In other embodiments, the test comprises at least one sequential figure recall moving display test module, which comprises target figures. In some embodiments, the test comprises at least one sequential word recall moving display test module and at least one sequential figure recall moving display test module.

Object Recall Spread Display Test Modules

Other test modules that challenge eye movement, particularly saccadic eye movement, include object recall spread display test modules. In these modules, eye movement and cognitive ability are challenged. Accordingly, in various embodiments, the test comprises one or more object recall spread display test modules. An object recall spread display test module comprises displaying a series of target objects to the subject, one object at a time, for a period of time (e.g., about 0.5, 1, 2, 3, 4, or 5 seconds). The target objects are rendered at random locations about the visual field of the display. For example, the objects may be rendered at the upper-left, upper-right, lower-left, lower-right, center-left, center-right, upper-center, lower-center, and center portions of the visual field. Typically, no two objects appear at the same locations in a given test module. After the target objects have been initially displayed, the target objects along with one or more distracter (i.e., non-target) objects are displayed statically in a group wherein the subject is requested to select the target objects among the objects displayed. The number of target objects selected, total number of target objects selected, number of times target or distracter items are selected more than once, number of distracter objects selected, number of selections made on neither a target object nor a distracter object (i.e., stray selections), total number of selections made, total number of selections less than or exceeding the number required to select all the target objects once, and the total time to complete the recall portion of the module may be recorded for scoring purposes.

In various embodiments, the object recall spread display test module comprises at least 2, 3, 4, 5, 6, 7, 8, 9, 10, 11, 12, 13, 14, or 15 target objects. In some embodiments, the object recall spread display test module comprises 4, 5, 6, 7, 8, 9, or 10 target objects (e.g., 5 or 6 target objects). In various embodiments, the object recall spread display test module comprises at least 2, 3, 4, 5, 6, 7, 8, 9, 10, 11, 12, 13, 14, or 15 distracter objects. In some embodiments, the object recall spread display test module comprises 4, 5, 6, 7, 8, 9, or 10 distracter objects (e.g., 5 or 6 distracter objects). In these and other embodiments, the number of distracter objects matches the number of target objects.

In various embodiments, the target and distracter objects are selected from the group consisting of letters, numbers, words, figures, pictures, shapes, and combinations thereof. Accordingly, in various embodiments, the test comprises one or more word recall spread display test modules, which comprise target words and distracter words. In other embodiments, the test comprises one or more figure recall spread display test modules, which comprise target figures and distracter figures. In some embodiments, the test comprises one or more word recall spread display test modules and one or more figure recall spread display test modules.

Sequential Recall Spread Display Test Modules

Further test modules that challenge eye movement, particularly saccadic eye movement, include sequential object recall spread display test modules. Similar to the object recall spread display test modules previously described, these modules challenge both eye movement and cognitive ability. Accordingly, in various embodiments, the test comprises one or more sequential object recall spread display test modules.

A sequential object recall spread display test module comprises displaying a series of target objects to the subject, one object at a time, for a period of time (e.g., about 0.5, 1, 2, 3, 4, or 5 seconds). The target objects are rendered at random locations about the visual field of the display. For example, the objects may be rendered at the upper-left, upper-right, lower-left, lower-right, center-left, center-right, upper-center, lower-center, and center portions of the visual field. Typically, no two objects appear at the same locations in a given test module. After the target objects have been initially displayed, the target objects are displayed statically in a group wherein the subject is requested to select the target objects in the order they were presented. The order the objects are selected, the number of objects selected, total number of selections less than or exceeding the number required to select all the target objects once, number of selections not in the correct order, number of times target items are selected more than once, number of selections not made on an object, total number of selections made, and the total time to complete the recall portion of the module may be recorded for scoring purposes.

In various embodiments, the sequential object recall spread display test module comprises at least 2, 3, 4, 5, 6, 7, 8, 9, 10, 11, 12, 13, 14, or 15 target objects. In some embodiments, the sequential object recall spread display test module comprises 4, 5, 6, 7, 8, 9, or 10 target objects (e.g., 5 or 6 target objects).

In various embodiments, the target objects are selected from the group consisting of letters, numbers, words, figures, pictures, shapes, and combinations thereof. Accordingly, in various embodiments, the test comprises one or more sequential word recall spread display test modules, which comprise target words. In other embodiments, the test comprises one or more sequential figure recall spread display test modules, which comprise target figures. In some embodiments, the test comprises one or more sequential word recall spread display test modules and one or more sequential figure recall spread display test modules.

Object Recall Fixed Display Test Modules

Other test modules that challenge eye movement, particularly saccadic eye movement, include object recall fixed display test modules. In these modules, eye movement and cognitive ability are challenged. Accordingly, in various embodiments, the test comprises one or more object recall fixed display test modules. An object recall fixed display test module comprises displaying a series of target objects to the subject, one object at a time, for a period of time (e.g., about 0.5, 1, 2, 3, 4, or 5 seconds). The target objects are rendered at single location of the visual field of the display (e.g., the center of the visual field of the display). After the target objects have been initially displayed, the target objects along with one or more distracter (i.e., non-target) objects are displayed statically in a group wherein the subject is requested to select the target objects among the objects displayed. The number of target objects selected, total number of target objects selected, number of times target or distracter items are selected more than once, number of distracter objects selected, number of selections made on neither a target object nor a distracter object (i.e., stray selections), total number of selections made, total number of selections less than or exceeding the number required to select all the target objects once, and the total time to complete the recall portion of the module may be recorded for scoring purposes.

In various embodiments, the object recall fixed display test module comprises at least 2, 3, 4, 5, 6, 7, 8, 9, 10, 11, 12, 13, 14, or 15 target objects. In some embodiments, the object recall fixed display test module comprises 4, 5, 6, 7, 8, 9, or 10 target objects (e.g., 5 or 6 target objects). In various embodiments, the object recall fixed display test module comprises at least 2, 3, 4, 5, 6, 7, 8, 9, 10, 11, 12, 13, 14, or 15 distracter objects. In some embodiments, the object recall fixed display test module comprises 4, 5, 6, 7, 8, 9, or 10 distracter objects (e.g., 5 or 6 distracter objects). In these and other embodiments, the number of distracter objects matches the number of target objects.

In various embodiments, the target and distracter objects are selected from the group consisting of letters, numbers, words, figures, pictures, shapes, and combinations thereof. Accordingly, in various embodiments, the test comprises one or more word recall fixed display test modules, which comprise target words and distracter words. In other embodiments, the test comprises one or more figure recall fixed display test modules, which comprise target figures and distracter figures. In some embodiments, the test comprises one or more word recall fixed display test modules and one or more figure recall fixed display test modules.

Sequential Recall Fixed Display Test Modules

Additional test modules that challenge eye movement, particularly saccadic eye movement, include sequential object recall fixed display test modules. Similar to the object recall fixed display test modules previously described, these modules challenge both eye movement and cognitive ability. Accordingly, in various embodiments, the test comprises one or more sequential object recall fixed display test modules.

A sequential object recall fixed display test module comprises displaying a series of target objects to the subject, one object at a time, for a period of time (e.g., about 0.5, 1, 2, 3, 4, or 5 seconds). The target objects are rendered at single location of the visual field of the display (e.g., the center of the visual field of the display). After the target objects have been initially displayed, the target objects are displayed statically in a group wherein the subject is requested to select the target objects in the order they were presented. The order the objects are selected, the number of objects selected, total number of selections less than or exceeding the number required to select all the target objects once, number of selections not in the correct order, number of times target items are selected more than once, number of selections not made on an object, total number of selections made, and the total time to complete the recall portion of the module may be recorded for scoring purposes.

In various embodiments, the sequential object recall fixed display test module comprises at least 2, 3, 4, 5, 6, 7, 8, 9, 10, 11, 12, 13, 14, or 15 target objects. In some embodiments, the sequential object recall fixed display test module comprises 4, 5, 6, 7, 8, 9, or target objects (e.g., 5 or 6 target objects).

In various embodiments, the target objects are selected from the group consisting of letters, numbers, words, figures, pictures, shapes, and combinations thereof. Accordingly, in various embodiments, the test comprises one or more sequential word recall fixed display test modules, which comprise target words. In other embodiments, the test comprises one or more sequential figure recall fixed display test modules, which comprise target figures. In some embodiments, the test comprises one or more sequential word recall fixed display test modules and one or more sequential figure recall fixed display test modules.

Object Recall Static Display Test Modules

Additional test modules that challenge eye movement, particularly saccadic eye movement, include object recall static display test modules. In various embodiments, the test comprises one or more object recall static display test modules. In general, an object recall static display test module comprises displaying a series of target objects as a group to the subject for a pre-defined period of time. After the target objects have been initially displayed, the target objects along with one or more distracter objects are displayed wherein the subject is requested to select the target objects among the group of objects displayed. The number of target objects selected, total number of target objects selected, number of times target or distracter items are selected more than once, number of distracter objects selected, number of selections made on neither a target object nor a distracter object (i.e., stray selections), total number of selections made, total number of selections less than or exceeding the number required to select all the target objects once, and the total time to complete the recall portion of the module may be recorded for scoring purposes.

In various embodiments, the object recall static display test modules comprise at least 2, 3, 4, 5, 6, 7, 8, 9, 10, 11, 12, 13, 14, or 15 target objects. In some embodiments, the object recall static display test modules comprise from 3 to 15, 4 to 14, 4 to 10, 5 to 15, 5 to 14, 5 to 10, or 5 to 8 target objects (e.g., 5 or 6 target objects). In various embodiments, the object recall static display test modules comprise at least 2, 3, 4, 5, 6, 7, 8, 9, 10, 11, 12, 13, 14, or 15 distracter objects. In some embodiments, the object recall static display test modules comprise 4, 5, 6, 7, 8, 9, or 10 distracter objects (e.g., 5 or 6 distracter objects). In these and other embodiments, the number of distracter objects matches the number of target objects.

In various embodiments, the target and distracter objects are selected from the group consisting of letters, numbers, words, figures, pictures, shapes, and combinations thereof. Accordingly, in various embodiments, the test comprises one or more word recall static display test modules, which comprise target words and distracter words. In other embodiments, the test comprises one or more figure recall static display test modules, which comprise target figures and distracter figures. In some embodiments, the test comprises one or more word recall static display test modules and one or more figure recall static display test modules.

In various embodiments, the target objects are initially displayed as a group for a pre-defined amount of time of about 30, 40, 50, or 60 seconds.

In various embodiments, the target objects and distracter objects are displayed after a short period of time has passed (e.g., about 0.5, 1, 2, 3, 4, 5, 10, 20, 30, 40, 50, or 60 seconds) subsequent to the initial display of the target objects (i.e., for immediate, short-term memory recall). In various embodiments, the target objects and distracter objects are displayed after a longer period of time has passed (e.g., at least about 5, 6, 7, 8, 9, or 10 minutes) subsequent to the initial display of the target objects (i.e., for delayed memory recall). For example, the target objects and distracter objects may be displayed for recall at or near the end of the test. Thus, the length of time between initial display and display for recall may be variable upon the speed at which a subject completes intervening test modules.

Permanent Memory Static Display Test Modules

Other test modules that challenge eye movement, particularly saccadic eye movement, include permanent memory static display test modules. In various embodiments, the test comprises one or more permanent memory static display test modules. In general, a permanent memory static display test module comprises displaying a group of target words that have a well-recognized association and/or order (e.g., months or weekdays) along with one or more distracter words (e.g., seasons, months, or weekdays, so long as the distracter words are not in the same category as the target words). The subject is requested to select the target words which fall in the requested category. The number of target words selected, total number of target words selected, number of times target or distracter items are selected more than once, number of distracter words selected, number of selections made on neither a target word nor a distracter word (i.e., stray selections), total number of selections less than or exceeding the number required to select all the target objects once, total number of selections made, and the total time to complete the module may be recorded for scoring purposes.

In various embodiments, the subject is requested to select the target words in their associated order or reverse order (e.g., Monday then Tuesday and so on; or Friday then Thursday and so on). Accordingly, in some embodiments permanent memory static display test modules comprise a sequential permanent memory static display test module. The order the target words are selected, the number of target words selected, total number of selections less than or exceeding the number required to select all the target objects once, number of selections not in the correct order, number of times target items are selected more than once, number of selections not made on a word (i.e., stray selections), total number of selections less than or exceeding the number required to select all the target objects once, total number of selections made, and the total time to complete the module may be recorded for scoring purposes.

In various embodiments, the permanent memory static display test modules comprise at least 2, 3, 4, 5, 6, 7, 8, 9, 10, 11, or 12 target objects. In some embodiments, the permanent memory static display test modules comprise from 3 to 15, 4 to 14, 4 to 10, 5 to 15, 5 to 14, 5 to 10, or 5 to 8 target objects (e.g., 5 or 6 target objects). In various embodiments, the permanent memory static display test modules comprise at least 2, 3, 4, 5, 6, 7, 8, 9, 10, 11, 12, 13, 14, or 15 distracter objects. In some embodiments, the permanent memory static display test modules comprise 4, 5, 6, 7, 8, 9, or 10 distracter objects (e.g., 5 or 6 distracter objects). In these and other embodiments, the number of distracter objects matches the number of target objects.

Other Modules

In addition to the one or more modules that challenge cognition and eye movement, the test may further comprise other test modules as needed, for example registration and/or pre-test questionnaire modules as outlined below.

Registration Module

In various embodiments, the test comprises a registration module. The registration module requests that the subject input applicable personal data such as:
First & Last Name
Height
Weight
Date of Birth
Gender
Handedness (Right or Left or Both)
Native Country
Language
Second Language
Years of Education
Association (school, university, or college name, business name, government entity, etc.)

Pre-Test Questionnaire Module

In various embodiments, the test comprises a pre-test questionnaire module. In various embodiments, the pre-test questionnaire module is typically displayed before other test modules besides the registration module. The pre-test questionnaire module requests that the subject input applicable injury data such as:
Purpose of Test—Practice or Normal
Have You Been Injured?
Injury Date & Time
Injury Location
Activity When Injured
Sport When Injured
Type of Vehicle
Automotive Restraints
Headgear Used
Other Conditions

### VIII. Scoring

In accordance with the present invention, following completion of a test or test module, one or more scores are computed as a function of the input received from the subject. In some embodiments, scores may be computed on a per module basis (module scores). For example, in an interceptor module, a score for the module may be computed based on the number of target objects intercepted by the subject-controlled object.

In various embodiments, scores may be computed based on subject input from two or more test modules which have similar test dimensions (dimension scores). Test modules having similar test dimensions include, for example, modules using similar target objects (e.g., two or more modules requiring the subject to select target words), modules challenging a subject to recall target objects in a similar timeframe (e.g., two or more modules measuring a subject's short-term recall), modules challenging a subject's eye tracking, and modules challenging a subject's hand-eye coordination.

In certain embodiments, scores may be computed based on subject input from two or more test modules of a test regardless of whether the test dimensions are the same (i.e., a total score). For example, a total score may be computed based on the total time a subject takes to complete a series test modules (e.g., all test modules of the test excluding administrative modules) or scores of a series of test modules (e.g., all test modules of the test excluding administrative and optionally game test modules such as an interceptor test module, avoidance test module, and/or the track test module).

In various embodiments, a combination of one or more module scores, one or more dimension scores, and one or more total scores are computed and provided to the subject.

### IX. Baseline Computation

In accordance with the present invention, at least one baseline is generated using scores from a plurality of previously completed tests (e.g., a subject's baseline). Generally, the plurality of previously completed tests useful for generating a baseline are obtained during a period of time when the subject is unimpaired/uncompromised, healthy (relatively healthy), or before the subject engages in an activity with the potential for causing impairment or compromise. For example, in the case of athlete subjects, a plurality of tests useful for generating a baseline may be collected during the off-season or just prior to the athletic season.

A baseline serves as a standard to which a subject's subsequent test scores may be compared to detect indicators of possible change in neuromechanical and neurocognitive function. The use of a plurality of tests in baseline computations differs from many prior neuropsychological and neurocognitive test methods, which use score(s) obtained from a single test completion for this purpose. Baselines in accordance with the present invention are statistical instruments that establish parameters of performance capability based on a documented, quantified performance history. To provide a valid and accurate estimate of a subject's characteristic performance capability, baselines must be computed using scores from a sufficient number of tests. However, establishing performance baselines representative of a subject's characteristic performance capability is not sufficient in and of itself when used for evaluation, screening, or other purposes. Correct statistical treatment and use of comparison data are required, as well. Typically, using the results of a single test as a baseline provides an extremely unreliable performance estimate while limiting the statistical methods that can be applied for subsequent performance comparison as described in the Background of the Invention section of this application.

In accordance with the present invention, a number of different baselines may be computed including a subject's recent baseline, a subject's extended baseline, a subject's composite baseline, and population baselines as outlined below.

Recent Baseline

In various embodiments, a subject's recent baseline is generated from the scores of a plurality of most recently completed qualifying tests. A pre-defined minimum number of qualifying tests may be required to generate a subject's baseline. For example, in various embodiments, a minimum of at least 2, at least 3, at least 4, at least 5, at least 6, at least 7, at least 8, at least 9, at least 10, at least 11, at least 12, at least 13, at least 14, or at least 15 previously completed qualifying tests by a subject are required to establish a subject's recent baseline. Accordingly, the subject's baseline is generated from a set of tests (“testing set”) that includes the minimum number of qualifying tests. In some embodiments, the minimum number of qualifying tests must be completed in a particular timeframe and/or according to a particular schedule. Additionally or alternatively, the subject may be limited to completing a subset of the testing set in a pre-defined amount of time. For example, at least 2, at least 3, at least 4, at least 5, at least 6, at least 7, at least 8, at least 9, at least 10, at least 11, at least 12, at least 13, at least 14, or at least 15 of the qualifying tests must each be completed by the subject on different days. As used herein, “qualifying tests” include tests that are fully completed by the subject (i.e., tests in which the subject only completes a portion of the test modules are not included), taken in normal mode (i.e., practice tests do not count), and satisfy other requirements according to various embodiments (e.g., only three tests completed on a given day count as “qualifying tests”).

In certain embodiments, a minimum of 2, a minimum of 3, a minimum of 4, a minimum of 8, a minimum of 12, a minimum of 16, a minimum of 20 qualifying tests, each completed by the subject on different days, is required to compute a subject's recent baseline. Using the minimum number of tests to compute a subject's recent baseline provides the statistical power necessary to minimize error and in particular reduce the probability of a false negative evaluation (i.e., an indication that no performance change has occurred when in fact it has).

Once a minimum number of qualifying tests have been completed, a subject's recent baseline may be generated by computing one or more mean scores (a measure of their average performance) and standard deviations (a measure of the amount of performance variation across the scores). Recent baselines may be computed based on individual test module scores, dimension scores, and/or total scores.

Extended Baseline

In various embodiments, a subject's extended baseline is generated from the scores of a subject's entire set of previously completed qualifying tests. A pre-defined minimum number of qualifying tests may be required to generate a subject's baseline. For example, in various embodiments, a minimum of at least 2, at least 3, at least 4, at least 5, at least 6, at least 7, at least 8, at least 9, at least 10, at least 11, at least 12, at least 13, at least 14, or at least 15 previously completed qualifying tests by a subject are required to establish a subject's extended baseline. Accordingly, the subject's baseline is generated from a set of tests (“testing set”) that includes the minimum number of qualifying tests. In some embodiments, the minimum number of qualifying tests must be completed in a particular timeframe and/or according to a particular schedule. Additionally or alternatively, the subject may be limited to completing a subset of the testing set in a pre-defined amount of time. For example, at least 2, at least 3, at least 4, at least 5, at least 6, at least 7, at least 8, at least 9, at least 10, at least 11, at least 12, at least 13, at least 14, or at least 15 of the qualifying tests must each be completed by the subject on different days.

Once a minimum number of tests have been completed, a subject's extended baseline may be determined by computing one or more mean scores (a measure of their average performance) and standard deviations (a measure of the amount of performance variation across the scores). Extended baselines may be computed based on individual test module scores, dimension scores, and/or total scores. In various embodiments, determining the extended baseline further comprises excluding from the computation at least a portion of the lowest scores from the plurality of scores obtained from previously completed qualifying tests. In some embodiments, the lowest 1%, 2%, 3%, 4%, 5%, 6%, 7%, 8%, 9% or 10% of scores are excluded from the mean score computation. For example, excluding the lowest 5% of scores eliminates all results that are two standard deviations or more below the baseline mean, as well as the lowest 2.6% of the scores that fall between the mean and −2 standard deviations. This approach takes into account the fact that many naturally occurring factors (illness, alcohol consumption, and abnormal hormone activity, for example) can negatively affect subject performance, while relatively few affect performance positively. In addition, extended baselines may be recomputed at the same time as recent baselines.

In certain embodiments, a minimum of 2, a minimum of 3, a minimum of 4, a minimum of 8, a minimum of 12, a minimum of 16, a minimum of 20 qualifying tests, each completed by the subject on different days, is required to compute a subject's extended baseline. Using the minimum number of tests to compute a subject's extended baseline provides the statistical power necessary to minimize error and in particular reduce the probability of a false negative evaluation (i.e., an indication that no performance change has occurred when in fact it has). In some embodiments, the minimum number of qualifying tests may be increased, to permit exclusion of the lowest score(s) from baseline computations without compromising the number of qualifying tests required for requisite statistical power.

Composite Baseline

In various embodiments, a subject's composite baseline is generated from the baseline parameters of a subject's entire set of computed recent baselines. Accordingly, the subject's composite baseline parameters are computed as the average of all recent baseline means and standard deviations for all baselines completed to date. Composite baselines may be computed based on individual test module scores, composite scores, and/or total scores. In addition, composite baselines may be recomputed at the same time as recent baselines.

Population Baseline

Population baselines are generated from a plurality of baseline parameters obtained from a plurality of subjects. A pre-defined minimum number of subjects may be required to compute a population baseline. In various embodiments, a relevant population baseline is determined for population groups of the same birth year and/or same gender. In various embodiments, determining a population baseline comprises excluding from the computation at least a portion of the lowest baseline parameters from the plurality of subjects. In some embodiments, the lowest 1%, 2%, 3%, 4%, 5%, 6%, 7%, 8%, 9% or 10% of subjects are excluded from the baseline parameter computation. For example, excluding the lowest 5% of baselines eliminates all subjects who are two standard deviations or more below the mean, as well as the lowest 2.6% of the subjects who fall between the mean and −2 standard deviations. This approach excludes the scores from subjects who are most severely affected by head blows or other impairing conditions they have experienced in the past. Thus, a population baseline may be determined by computing an average mean (excluding a certain portion of the lowest scores) and an average standard deviation using the included subjects' extended and or composite baseline parameters. Population baselines may be computed based on individual test module scores, dimension scores, and/or total scores. In addition, population baselines may be recomputed as needed, for example on a weekly basis.

### X. Score Comparison

In accordance with the present invention, the subject scores are compared to at least one baseline. In various embodiments, the subject scores are compared to at least one subject baseline. In certain embodiments, the subject baseline is selected from the group consisting of a subject's recent baseline, a subject's extended baseline, a subject's composite baseline, and combinations thereof. In these and other embodiments, the subject scores are also compared a population baseline (e.g., a relevant population baseline). In some embodiments, the subject scores are compared to a subject's recent baseline, a subject's extended baseline, and a relevant population baseline.

In various embodiments, the process of comparing a score to a baseline comprises computing the number of baseline standard deviations the subject score falls from the baseline mean score (i.e., standard deviation offset). Standard deviation offset is computed according to formula (1) when a higher score is indicative of higher performance (e.g., interceptor module score):

$$Offset = \frac{( X - \overline{X} )}{σ} \qquad ( 1 )$$

Standard deviation offset is computed according to formula (2) when a lower score is indicative of higher performance (e.g., avoidance module score):

$$Offset = \frac{( \overline{X} - X )}{σ} \qquad ( 2 )$$

In formulas 1 and 2, “X” is a score (e.g., a module, dimension, or total score), X-bar is the mean score of the baseline, and σ is the standard deviation.

In some embodiments, the process of comparing a score to a baseline comprises assigning a score to a standard deviation zone based on its standard deviation offset.

In some embodiments, letters of the alphabet may be used as designators for standard deviation zones. Thus, for example, the letters A through F might be used if there are six zones, A through H for eight zones, A through J for ten zones, and so forth.

In some embodiments, standard deviation offsets and standard deviation zone assignments may be stored in the computer-readable medium along with other test data.

In various embodiments, providing the score comparison for displaying to the subject comprises generating a color-coded result which represents the score comparison and a color-code key which provides the meaning of the result.

### XI. Indicators of Impairment or Improvement

In accordance with the present invention, statistical process control (SPC) methods can be used effectively to monitor human physical and mental processes. SPC methods have been used in a wide variety of industrial applications (e.g., manufacturing and software development), but they have not been applied previously to neuropsychological, neurocognitive, neuromechanical, or other similar testing, evaluation, and/or screening. Human physical and mental processes typically are not as predictable as industrial processes, nor can they be measured as precisely. Consequently, applicants have adapted existing SPC methods appropriately for use in monitoring neuromechanical and neurocognitive function. As adapted, SPC methods provide a set of sensitive indicators of subject impairment and/or improvement.

Applying SPC to human processes requires that subject baselines be established using a plurality of data points to enable computation of mean and standard deviation values specific to each individual subject. These values quantify the expected level and variability of each subject's performance based on their demonstrated performance history. “Common causes” of variation (i.e., elements that are inherent in the processes themselves) are automatically considered in baseline computations. Common causes of variation in human test performance include, for example, the subject's natural variation from one test to another, reliability of the test and/or testing process, environmental factors, and other routinely occurring events or conditions for that subject.

When baseline parameters include the effects of common cause variation, subsequent subject performance will reflect the presence of “special” (or “attributable”) causes of variation. Such conditions as closed cranial injuries (even if they do not qualify as concussions), fatigue, drug or alcohol use, and abnormal blood sugar levels result in performance values and/or patterns that indicate, for example, a negative shift from baseline parameters. The results of on-going subject performance testing can be monitored and evaluated as needed to detect the presence of existing or developing impairment, even when the special causes themselves are unsuspected or unknown.

Post-baseline performance monitoring also can be used to detect improvement when baselines are established for subjects who are already impaired. Impairment evaluations can be applied in reverse to detect indications of a positive shift from baseline parameters.

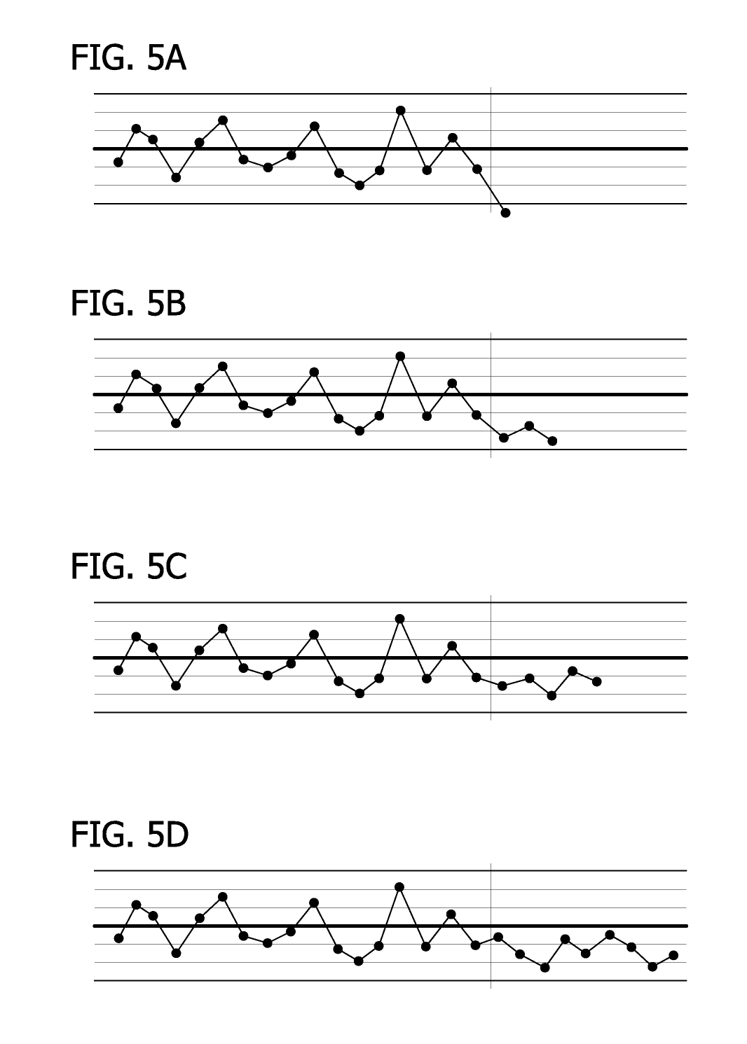

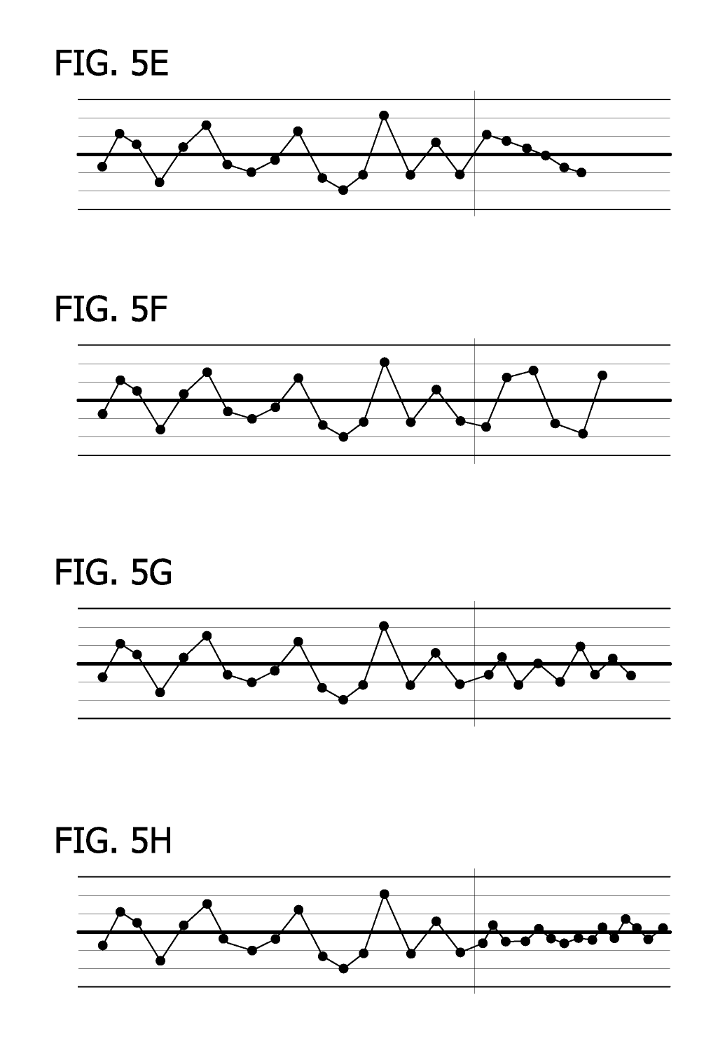

At least eight rules or approaches of SPC methods may be applied to detect indicators of impairment or improvement from baseline parameters. These rules are illustrated in FIGS. 5A-5H. In these figures the baseline mean is represented by the center horizontal line. The horizontal lines above and below the center line illustrate the number of standard deviations above or below the mean (i.e., +3σ, +2σ, +1σ, mean, −1σ, −2σ, −3σ, respectively). The vertical line on the graph indicates the point at which the baselines are computed. Test scores to the left of this line are used for computing baselines while those to the right are used for evaluation of performance relative to the statistical baseline parameters. As used herein, “σ” refers to a standard deviation (e.g., subject or population).

One approach that indicates possible impairment or improvement determines if a single score is three or more standard deviations from the mean. For example, see FIG. 5A which shows a graph of a series of test scores illustrating a test score falling outside of three standard deviations of the baseline. The probability that a score meeting this approach would be the result of chance instead of impairment or improvement is no greater than about 0.1%. Accordingly, in various embodiments, the computer-executed method, system, and computer-readable medium further comprise applying (or memory storing computer executable instructions for applying or computer executable instructions for applying) a statistical process control method to one or more subject score comparisons; generating an indication of subject impairment or improvement based on the applying; and providing for display to the subject the generated indication by determining whether a single score is three standard deviations or more from the mean score.

A second approach that indicates possible impairment or improvement determines if two out of three scores (e.g., corresponding module score(s), composite score(s), and/or total score(s)) from three or more of a subject's tests are two or more standard deviations from the mean. For example, see FIG. 5B which shows a graph of a series of test scores illustrating two test scores out of three falling outside of two standard deviations of the mean. The probability that a score meeting this second approach would be the result of chance instead of impairment or improvement is no greater than about 0.14%. Accordingly, in various embodiments, the computer-executed method, system, and computer-readable medium further comprise applying (or memory storing computer executable instructions for applying or computer executable instructions for applying) a statistical process control method to one or more subject score comparisons; generating an indication of subject impairment or improvement based on the applying; and providing for display to the subject the generated indication by determining whether two scores relating to the same test module or collection of test modules obtained during the course of a subject completing three consecutive tests are two or more standard deviations from the mean score.

A third approach that indicates possible impairment or improvement determines is four out of five scores (e.g., corresponding module score(s), composite score(s), and/or total score(s)) obtained from five or more of a subject's tests are more than one standard deviation from the mean. For example, see FIG. 5C which shows a graph of a series of test scores illustrating four test scores out of five falling outside of one standard deviation of the mean. The probability that a score meeting this third approach would be the result of chance instead of impairment or improvement is no greater than about 0.22%. Accordingly, in various embodiments, the computer-executed method, system, and computer-readable medium further comprise applying (or memory storing computer executable instructions for applying or computer executable instructions for applying) a statistical process control method to one or more subject score comparisons; generating an indication of subject impairment or improvement based on the applying; and providing for display to the subject the generated indication by determining whether four scores relating to the same test module or collection of test modules obtained during the course of a subject completing five consecutive tests are more than one standard deviation from the mean score.

A fourth approach that indicates possible impairment or improvement determines if nine consecutive scores (e.g., corresponding module score(s), composite score(s), and/or total score(s)) obtained from nine or more of a subject's tests are on the same side of the mean. For example, see FIG. 5D which shows a graph of a series of test scores illustrating nine consecutive test scores falling on one side of the mean. The probability that a score meeting this fourth approach would be the result of chance instead of impairment or improvement is no greater than about 0.19%. Accordingly, in various embodiments, the computer-executed method, system, and computer-readable medium further comprise applying (or memory storing computer executable instructions for applying or computer executable instructions for applying) a statistical process control method to one or more subject score comparisons; generating an indication of subject impairment or improvement based on the applying; and providing for display to the subject the generated indication by determining whether nine scores relating to the same test module or collection of test modules obtained during the course of a subject completing nine consecutive tests are on the same side of the mean score.

A fifth approach that indicates possible developing impairment or improvement determines if six consecutive scores (e.g., corresponding module score(s), composite score(s), and/or total score(s)) obtained from a subject's six consecutive tests all are increasing or decreasing (i.e., drifting upward or drifting downward). For example, see FIG. 5E which shows a graph of a series of test scores illustrating six test scores in a row in a decreasing trend. A downward drifting trend provides an indication of gradually increasing impairment. Conversely, an upward drifting trend provides an indication of gradual improvement. The probability that a score meeting this fifth approach would be the result of chance instead of consistent impairment or improvement is no greater than about 1.6%. Accordingly, in various embodiments, the computer-executed method, system, and computer-readable medium further comprise applying (or memory storing computer executable instructions for applying or computer executable instructions for applying) a statistical process control method to one or more subject score comparisons; generating an indication of subject impairment or improvement based on the applying; and providing for display to the subject the generated indication by determining whether six scores relating to the same test module or collection of test modules obtained during the course of a subject completing six consecutive tests demonstrate a decreasing or increasing trend.

A sixth approach that indicates possible impairment determines if six consecutives scores (scores relating to the same module score, composite score, and/or total score) obtained from a subject's six consecutive tests indicate a consistently mixed pattern of relatively extreme high and low values (e.g., a mixture of scores below −1σ and above +1σ). For example, see FIG. 5F which shows a graph of a series of test scores illustrating six test scores in a row having mixed high and low values. A mixed high and low value trend provides an indication of erratic performance which may reflect a temporary and/or intermittent impairment caused by, for example, use of substances like methamphetamines that cause extreme shifts in behavior. The probability that a set of scores meeting this sixth approach would be the result of chance instead of impairment is no greater than about 0.1%. Accordingly, in various embodiments, the computer-executed method, system, and computer-readable medium further comprise applying (or memory storing computer executable instructions for applying or computer executable instructions for applying) a statistical process control method to one or more subject score comparisons; generating an indication of subject impairment or improvement based on the applying; and providing for display to the subject the generated indication by determining whether six scores relating to the same test module or collection of test modules obtained during the course of a subject completing six consecutive tests demonstrate a mixed high and low value trend.

A seventh approach determines if nine consecutives scores (scores relating to the same module score, composite score, and/or total score) obtained from a subject's nine consecutive tests indicate an alternating trend (e.g., scores alternating up and down). For example, see FIG. 5G which shows a graph of a series of test scores illustrating nine test scores in a row in an alternating trend. An alternating trend provides an indication of erratic performance which may reflect a temporary and/or intermittent impairment caused by, for example, substance use. The probability that a score meeting this seventh approach would be the result of chance instead of impairment is no greater than about 0.1%. Accordingly, in various embodiments, the computer-executed method, system, and computer-readable medium further comprise applying (or memory storing computer executable instructions for applying or computer executable instructions for applying) a statistical process control method to one or more subject score comparisons; generating an indication of subject impairment or improvement based on the applying; and providing for display to the subject the generated indication by determining whether nine scores relating to the same test module or collection of test modules obtained during the course of a subject completing nine consecutive tests demonstrate an alternating trend.

An eighth approach determines if 15 consecutives scores (scores relating to the same module score, composite score, and/or total score) obtained from the subject's 15 consecutive tests indicate a trend of increased performance consistency (e.g., scores falling between plus one and minus one standard deviations from the mean). For example, see FIG. 5H which shows a graph of a series of test scores illustrating 15 test scores in a row falling between plus one and minus one standard deviations from the mean. The probability that a score meeting this eighth approach would be the result of chance instead of consistent improvement is no greater than about 0.3%. Accordingly, in various embodiments, the computer-executed method, system, and computer-readable medium further comprise applying (or memory storing computer executable instructions for applying or computer executable instructions for applying) a statistical process control method to one or more subject score comparisons; generating an indication of subject impairment or improvement based on the applying; and providing for display to the subject the generated indication by determining whether 15 scores relating to the same test module or collection of test modules obtained during the course of a subject completing 15 consecutive tests demonstrate an alternating trend.

In various embodiments of the present invention, the statistical trends of a subject's score in relation to a baseline outlined above are used for generating an indication of impairment and/or improvement. In various embodiments, one or more of these approaches is used to detect indicators of possible impairment and/or improvement. In some embodiments, all of these approaches are used independently or in combination to detect indicators of possible impairment and/or improvement.

In addition to the evaluation approaches described above, the use of a plurality of tests for computing baseline parameters allows statistical baseline comparison of any two or more of a subject's baselines to determine the statistical significance of any differences between values using analysis of variance (ANOVA) and other well-established experimental research approaches. This kind of comparison provides a means for detecting impairment or improvement trends over extended time periods.

In some embodiments, providing the indication of impairment or improvement for displaying to the subject comprises generating a color-coded result which represents the indication and a color-code key which provides the meaning of the result. For example, one color-coded scheme may comprise the following color-coded results:
Color 1 (e.g., green): the criteria have not been satisfied, indicating performance across the tests used for evaluation is clearly consistent with baseline performance;
Color 2 (e.g., yellow): the criteria have not been satisfied but performance across the tests used for evaluation is approaching satisfaction, indicating possible onset of impairment;
Color 3 (e.g., aqua): the criteria have not been satisfied but performance across the tests used for evaluation is approaching satisfaction, indicating possible onset of improvement;
Color 4 (e.g., orange): the criteria have been at least minimally satisfied and performance across the tests used for evaluation clearly departs from baseline performance, indicating existence of possible impairment;
Color 5 (e.g., blue): the criteria have been at least minimally satisfied and performance across the tests used for evaluation clearly departs from baseline performance, indicating existence of possible improvement;
Color 6 (e.g., red): the criteria have been substantially exceeded and performance across the tests used for evaluation departs substantially from baseline performance, indicating existence of possible significant impairment;
Color 7 (e.g., purple): the criteria have been substantially exceeded and performance across the tests used for evaluation departs substantially from baseline performance, indicating existence of possible significant improvement;
Color 8 (e.g., gray): status is indeterminate from the pattern displayed by performance across the tests used for evaluation; and
Color 9 (e.g., white): the available performance data are insufficient to apply the evaluation approach.

### Example

The following non-limiting example is provided to further illustrate the present invention.

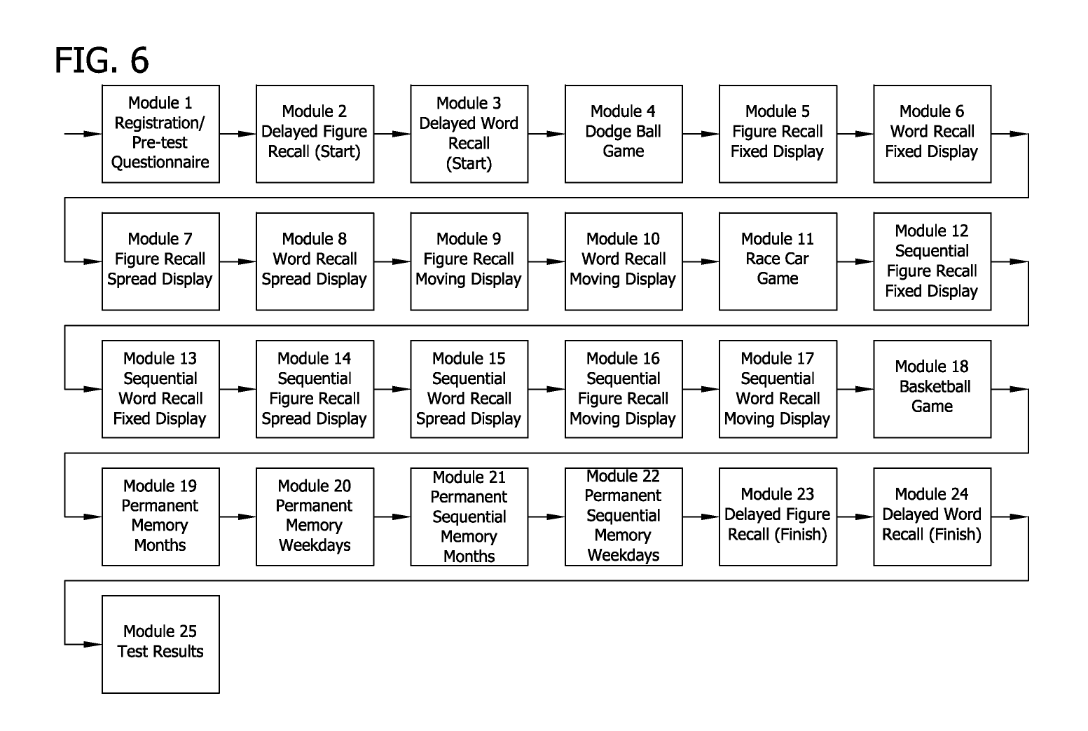

FIG. 6 depicts a computer- or web-based test of one embodiment of the present invention, which features a series of test modules. The test includes 25 test modules which are rendered sequentially for subject input or display to the subject.

Module 1 is a Registration/Pre-test Questionnaire module which requests a subject to input various personal information, information relating to any injuries, etc.

Modules 2 and 3 are the initial display portions of the Delayed Figure and Word Recall (Static Display) modules, respectively. In these modules, a group of figures and a group of words are rendered (displayed) to the subject as previously described herein in the Object Recall Static Display Test Modules section.

Module 4 is a Dodge Ball Game which is an example of an avoidance test module as previously described herein. FIG. 3 provides an illustrative screen shot of the Dodge Ball Game.

Modules 5 and 6 are Figure and Word Recall Fixed Display modules, respectively. These modules are described above in the Object Recall Fixed Display Test Modules section.

Modules 7 and 8 are Figure and Word Recall Spread Display modules, respectively. These modules are described above in the Object Recall Spread Display Test Modules section.

Modules 9 and 10 are Figure and Word Recall Moving Display modules, respectively. These modules are described above in the Object Moving Display Test Modules section.

Module 11 is a Race Car Game which is an example of a track test module as previously described herein. FIG. 4 provides an illustrative screen shot of the Race Car Game.

Modules 12 and 13 are Sequential Figure and Word Recall Fixed Display modules, respectively. These modules are described above in the Sequential Object Fixed Display Test Modules section.

Modules 14 and 15 are Sequential Figure and Word Recall Spread Display modules, respectively. These modules are described above in the Sequential Object Spread Display Test Modules section.

Modules 16 and 17 are Sequential Figure and Word Recall Moving Display modules, respectively. These modules are described above in the Sequential Object Recall Moving Display modules section.

Module 18 is a Basketball Game which is an example of an interceptor test module as previously described herein. FIG. 2 provides an illustrative screen shot of the Basketball Game.

Modules 19, 20, 21, and 22 are Permanent Memory Modules for months, weekdays, sequential months and sequential weekdays, respectively. These modules are described above in the Permanent Memory Static Display Test Modules section.

Modules 23 and 24 are the recall portion of the Delayed Figure and Word Recall Modules (Modules 1 and 2). These modules are described above in the Object Recall Static Display Test Modules section.

Module 25 provides the test results for display to the subject, including various test module score(s), dimension score(s), and total score(s) in addition to various comparisons to one or more baselines.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above method, system, and computer readable medium without departing from the scope of the invention, it is intended that all matter contained in the above description and shown in the accompanying drawing[s] shall be interpreted as illustrative and not in a limiting sense. Having described the invention in detail, it will be apparent that modifications and variations are possible without departing from the scope of the invention defined in the appended claims.

1. A computer-executed method for testing neuromechanical and neurocognitive function in a subject comprising:
rendering a test comprising at least one test module for displaying the test to the subject;
generating a request for subject input indicative of the subject's response to the displayed test;
receiving the subject input indicative of the subject's response to the test;
computing at least one subject score as a function of the received subject input;
comparing the at least one subject score to at least one baseline comprising a subject baseline, wherein the subject baseline comprises a mean score and a standard deviation computed from a plurality of scores obtained from previously completed tests by the subject; and
providing the at least one subject score and providing the score comparison for displaying to the subject.

2. The method of claim 1 wherein the test comprises two or more test modules and wherein the method comprises:
computing at least one module score, at least one dimension score, and at least one total score as a function of the received subject input;
comparing the at least one module score, at least one dimension score, and at least one total score to at least one baseline; and
providing the computed scores and the score comparison for displaying to the subject.

3. The method of claim 1 wherein comparing the at least one subject score comprises computing the number of baseline standard deviations the score falls from the baseline mean score (standard deviation offset).

4. The method of claim 1 wherein the subject baseline is selected from the group consisting of a subject's recent baseline, a subject's extended baseline, a subject's composite baseline, and combinations thereof.

5. The method of claim 1, wherein the subject baseline is generated by computing a mean score and standard deviation from a plurality of scores obtained from at least 3, at least 4, at least 5, at least 6, at least 7, at least 8, at least 9, at least 10, at least 11, at least 12, at least 13, at least 14, or at least 15 previously completed tests by the subject.

6. The method of claim 1 wherein the at least one subject score is compared to a population baseline.

7. (canceled)

8. The method of claim 6 wherein the at least one subject score is compared to a subject's recent baseline, a subject's extended baseline, and a relevant population baseline.

9-13. (canceled)

14. The method of claim 1 further comprising applying a statistical process control method to one or more subject score comparisons; generating an indication of subject impairment or improvement based on the applying; and providing for display to the subject the generated indication.

15. The method of claim 14 wherein applying a statistical process control method comprises applying one or more approaches comprising:
(1) determining whether a single score (module score, composite score, and/or total score) is three or more standard deviations from the mean score,
(2) determining whether two out of three consecutive scores (corresponding module scores, composite scores and/or total scores) obtained from three or more of the subject's tests are two or more standard deviations from the mean score on the same side,
(3) determining whether four out of five consecutive scores (e.g., corresponding module score(s), composite score(s), and/or total score(s)) obtained from five or more of the subject's tests are more than one standard deviation from the mean score on the same side,
(4) determining whether nine consecutive scores (e.g., corresponding module score(s), composite score(s), and/or total score(s)) obtained from nine or more of a subject's tests are on the same side of the mean score,
(5) determining whether six consecutive scores (e.g., corresponding module score(s), composite score(s), and/or total score(s)) obtained from the subject's six consecutive tests all are increasing or decreasing,
(6) determining whether six consecutive scores (scores relating to the same module score, composite score, and/or total score) obtained from the subject's six consecutive tests indicate a consistent mixed pattern of relatively extreme high and low values by falling more than one standard deviation from the mean score on both sides,
(7) determining whether nine consecutive scores (scores relating to the same module score, composite score, and/or total score) obtained from the subject's nine consecutive tests indicate a consistent alternating trend (e.g., scores alternating up and down), and/or
(8) determining whether 15 consecutives scores (scores relating to the same module score, composite score, and/or total score) obtained from the subject's 15 consecutive tests indicate a trend of increased performance consistency by falling between plus one and minus one standard deviations from the mean score.

16. The method of claim 1 wherein the test comprises at least 3, at least 4, at least 5, at least 6, at least 7, at least 8, at least 9, at least 10, at least 15, at least 20 or at least 25 test modules.

17. (canceled)

18. The method of claim 16 wherein the test comprises at least 3, at least 4, at least 5, at least 6, at least 7, at least 8, at least 9, at least 10, at least 15, at least 20 or at least 25 test modules that challenge eye movement.

19. (canceled)

20. The method of claim 1 wherein the test comprises at least one test module for displaying to the subject selected from the group consisting of an interceptor test module, an avoidance test module, a track test module, and combinations thereof.

21-27. (canceled)

28. The method of claim 1 wherein subject input is received by an input device selected from the group consisting of a computer mouse, keyboard, touch-screen, touch-pad, joystick including an isometric joystick, remote control, a gaming controller, a motion sensing input device and combinations thereof.

29. The method of claim 1 wherein the test is displayed to the subject on an electronic viewing device selected from the group consisting of a computer monitor, touch-screen, television, electronic tablet device, hand-held gaming device, and wireless phone.

30. The method of claim 1 wherein the computer-executed method is for testing neuromechanical and neurocognitive function which reflects physical and mental compromise associated with traumatic brain injury and other closed cranial injury in a subject.

31. The method of claim 1 wherein the computer-executed method is for testing neuromechanical and neurocognitive function which reflects physical and mental compromise associated with impairment to brain function caused by substance use or abuse, diseases including nervous system diseases, disorders which alter brain activity, psychiatric disorders, conditions which impact brain function, or accidental or naturally-occurring events which impact brain function.

32. The method of claim 1 wherein the computer-executed method is for testing neuromechanical and neurocognitive function which reflects the physical and mental readiness of a subject prior to, during, and/or subsequent to engaging in the subject's occupation.

33. The method of claim 1 further comprising:
generating a request for subject-specific information from the subject;
receiving the subject-specific information;
rendering a test comprising at least one test module based on the subject-specific information received for displaying to the subject;
generating a request for subject input indicative of the subject's response to the test module;
receiving subject input indicative of the subject's response to the test module;
computing a subject score as a function of the received subject input;
comparing the subject score to a baseline; and
providing the subject score and the score comparison for displaying to the subject.

34. A system for testing neuromechanical and neurocognitive function in a subject comprising:
a memory storing instructions for a test comprising at least one test module, subject input, subject scores, and at least one subject baseline and
a processor configured to execute computer executable instructions comprising:
rendering a test comprising at least one test module for displaying the test to the subject;
generating a request for subject input indicative of the subject's response to the displayed test;
receiving the subject input indicative of the subject's response to the displayed test;
computing at least one subject score as a function of the received subject input;
comparing the at least one subject score to at least one subject baseline, wherein the at least one subject baseline comprises a mean score and a standard deviation computed from a plurality of scores obtained from previously completed tests by the subject; and
providing the at least one subject score for displaying to the subject and providing the score comparison for displaying to the subject.

35-66. (canceled)

67. A computer-readable tangible non-transitory medium storing instructions for testing neuromechanical and neurocognitive function in a subject comprising computer executable instructions for:
rendering a test comprising at least one test module for displaying the test to the subject;
generating a request for subject input indicative of the subject's response to the displayed test;
receiving the subject input indicative of the subject's response to the displayed test;
computing at least one subject score as a function of the received subject input;
comparing the at least one subject score to at least one subject baseline, wherein the at least one subject baseline comprises a mean score and a standard deviation computed from a plurality of scores obtained from previously completed tests by the subject; and
providing the at least one subject score for displaying to the subject and providing the score comparison for displaying to the subject.

68-97. (canceled)
